(19) United States
(12) Patent Application Publication
(10) Pub. No.: US 2016/0239618 A1
(43) Pub. Date: Aug. 18, 2016

(54) BODILY FLUID ANALYZER WITH SELF-DIAGNOSIS FUNCTION AND BODILY FLUID ANALYZING SYSTEM COMPRISING THE SAME

(71) Applicant: BBB INC., Seongnam-si (KR)

(72) Inventors: Jaekyu CHOI, Seongnam-si (KR); Jisoon LIM, Seongnam-si (KR)

(21) Appl. No.: 14/852,349

(22) Filed: Sep. 11, 2015

(30) Foreign Application Priority Data
Feb. 13, 2015 (KR) 10-2015-0022282

(51) Int. Cl. G06F 19/00; G01N 33/49

(52) U.S. Cl. CPC G06F 19/345; G01N 33/49; G06F 19/366; G06F 19/3406

(57) ABSTRACT

The present disclosure relates to bodily fluid analyzer with self-diagnosis function comprising, the first sensor part and the first operation part that are used to test or analyze the bodily fluid sample; the second sensor part and the second operation part that are used to self-diagnose of abnormalities of the said first sensor part or the first operation part; and the control part that operates the first operation part or the second operation part based on the predefined conditions and senses the operation status of the first operation part or the second operation part by using the second sensor part or the first sensor part, wherein the first and second sensor parts are used as a means of data entry into the aforementioned bodily fluid analyzer and the said first and second operation parts are used as means of output of the bodily fluid analyzer.

### CROSS-REFERENCE TO RELATED APPLICATION

This application claims the priority of the Korean Patent Application No. 10-2015-0022282, filed on Feb. 13, 2015 in the Korean Intellectual Property Office (KIPO), and all the benefits accruing therefrom under 35 U.S.C. §119, the contents of which in their entirety are herein incorporated by reference.

### TECHNICAL FIELD

This invention relates to a bodily fluid analyzer with self-diagnosis function and a bodily fluid analyzing system comprising the same and more specifically, a bodily fluid analyzer with self-diagnosis function that is based on a mobile OS (Operation System) and a bodily fluid analyzing system comprising the same.

### BACKGROUND ART

There is a surge in interest in health care due to increased life expectancy, which has drawn attention to the healthcare industry. Diverse pieces of apparatus that enable health care services were already developed or are being developed and the existing apparatus is being enhanced in a user-friendly way.

One of the representative pieces of health care apparatus is a blood analyzer and R&D of this is being conducted continuously because blood analysis helps us obtain useful health information. Specifically, the current blood analyzer can display the result of blood test on the analyzer and has user interface to an extent that it can print out the test result if necessary.

### DISCLOSURE OF INVENTION

### Technical Problem

However, the existing blood analyzer is designed based on firmware without OS or even if it has an embedded OS, it is designed as an embedded system in no consideration of connectivity to external network.

Therefore, the user of a blood analyzer has difficulty providing objective status information of the blood analyzer for an expert in case the blood analyzer has a problem. It is required for an expert to examine in person and repair the blood analyzer offline in order to resolve such a problem, which would cause inconvenience to the user.

Based on the problems mentioned above, the technical issue that the present invention is to resolve is to provide a bodily fluid analyzer with self-diagnosis function and a bodily fluid analyzing system comprising the same that enables the collection of objective information about the status of the blood analyzer via self-diagnosis.

Another technical issue of this invention is to provide a bodily fluid analyzer with self-diagnosis function and a bodily fluid analyzing system comprising the same that allows the objective information collected via self-diagnosis about the blood analyzer to be sent out over the network.

The technical issues the present invention is to resolve are not limited to the above mentioned, but other issues not mentioned herein will be clearly understandable for those having ordinary skill in the art based upon the description which follows.

### Effects of Invention

According to the present invention mentioned above, the following effects are expected. However, the effects that can be obtained from this invention are not limited to the effects stated below.

First, according to the present invention, objective information about the status of the blood analyzer can be collected via self-diagnosis. In other words, not only a simple indication of a problem with the blood analyzer but specific information that notifies an area of the problem in the blood analyzer components can be obtained.

Second, this invention can enable connectivity to external devices or networks because it is based on a mobile OS, comprising a communication module. Therefore, according to the present invention, problem solving of the blood analyzer can be done remotely.

### BRIEF DESCRIPTION OF DRAWINGS

FIG. 1 is a drawing illustrating an outline of composition of the blood analyzer system in accordance with one exemplary embodiment of the present invention.

FIG. 2 is a drawing illustrating an outline of the blood analyzer in accordance with one exemplary embodiment of the present invention.

FIG. 3 is a drawing illustrating an outline of composition of the management server in accordance with one exemplary embodiment of the present invention.

FIG. 4 is a drawing illustrating an outline of composition of the blood analyzer in accordance with another exemplary embodiment of the present invention.

### DETAILED DESCRIPTION OF THE INVENTION

A desirable exemplary embodiment of the present invention will be described in detail hereinafter in reference with the accompanied drawings. The advantage and characteristic, and a method to achieve them will be obvious upon detailed description of the exemplary embodiments as well as accompanied drawings which follow. However, the present invention is not limited to the exemplary embodiments set forth herein, but can be embodied in many different forms. The exemplary embodiments are provided only to render the present disclosure complete and the scope of the present invention comprehensible to those having ordinary skill in the art. The present invention is only to be defined by the claims. A same reference mark used refers to a same element throughout the description.

Without separate definitions, all terms (including technical and scientific terms) used in the present description may be used for the meanings commonly understandable to those having ordinary skill in the art. In addition, the terms generally used and having definitions in dictionary, unless otherwise defined obviously in particular, should not be ideally or exaggeratedly interpreted.

The terms used in the present description are to explain the exemplary embodiments, not to limit the present invention thereto. In the present description, a singular form of word also includes a plural form thereof unless otherwise noted. The term “comprises” and/or “comprising” is not excluding the meaning that one or more elements other than the said element may exist or be added.

The bodily fluid analyzer with self-diagnosis function and the bodily fluid analyzing system comprising the same in accordance with exemplary embodiments of the present invention are described hereinafter in reference with the accompanied drawings. Although the bodily fluid analyzer and the bodily fluid analyzing system in accordance with exemplary embodiments of the present invention can perform basic function to test and analyze samples of bodily fluid, this specification focuses on self-diagnosis function apart from the basic function.

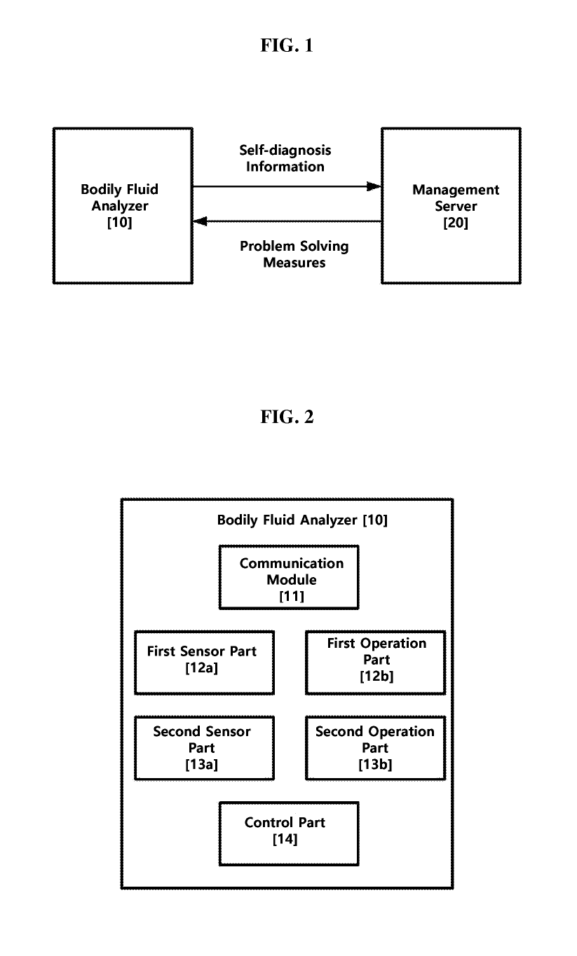

First, the bodily fluid analyzing system in accordance with one exemplary embodiment of the present invention is described in reference with FIG. 1. FIG. 1 shows an outline of composition of the bodily fluid analyzing system in accordance with one exemplary embodiment of the present invention.

According to FIG. 1, the bodily fluid analyzing system in accordance with one exemplary embodiment of the present invention may comprise a bodily fluid analyzer (10) with self-diagnosis function and a management server (20). The bodily fluid may refer to but is not limited to blood, lymph fluid, and tissue fluid.

The bodily fluid analyzer (10) is a device that can receive the bodily fluid sample and test and analyze the characteristics of it, providing useful health information about the supplier of the bodily fluid sample based on the test and analysis of the bodily fluid sample.

Moreover, the bodily fluid analyzer (10) included in the bodily fluid analyzing system in accordance with one exemplary embodiment of the present invention can have self-diagnosis function. In other words, before testing and analyzing the bodily fluid sample, the bodily fluid analyzer (10) can self-diagnose to check whether it malfunctions or not. The specific composition for self-diagnosis function of the bodily fluid analyzer (10) will be described later.

After the bodily fluid analyzer (10) self-diagnoses, it can send the result of self-diagnosis to the management server (20). Here, the result of self-diagnosis may include not only the indication of an abnormality in the bodily fluid analyzer (10) but also more of test or analysis results obtained from self-diagnosis.

The management server (20) can be, for example, a cloud server but the type of the management server (20) is not limited thereto. The management server (20) enables diverse functions like receiving the test and analysis results of the bodily fluid sample from the bodily fluid analyzer (10) and managing such information but in this specification, the focus of function descriptions is the feedback given by the management server (20) to the bodily fluid analyzer (10) corresponding to the self-diagnosis function of the bodily fluid analyzer (10).

The management server (20) can receive the self-diagnosis result from the bodily fluid analyzer (10) and send problem solving measures regarding the received self-diagnosis result to the bodily fluid analyzer (10). In other words, the management server (20) can analyze the result of self-diagnosis received from the bodily fluid analyzer (10), indicate a problem with the bodily fluid analyzer (10), and send problem solving measures required to resolve the issue with the bodily fluid analyzer (10) to the bodily fluid analyzer (10).

If the management server (20) concludes that the issue with the bodily fluid analyzer (10) can be resolved by the user of the bodily fluid analyzer (10), the problem solving measures may include guidance information that helps the user of the bodily fluid analyzer (10) operate the bodily fluid analyzer (10) based on the problem solving measures. For example, the problem solving measures that are provided for the bodily fluid analyzer (10) can be a kind of UX (User Experience) and the user of the bodily fluid analyzer (10) can be guided to take appropriate measures against the bodily fluid analyzer (10) according to the problem solving measures.

If the management server (20) concludes that the issue with the bodily fluid analyzer (10) cannot be addressed by the user of the bodily fluid analyzer (10), the problem solving measures may include but is not limited to guidance information that helps the user of the bodily fluid analyzer (10) report the failure of the bodily fluid analyzer (10) based on the problem solving measures. For example, the problem solving measures may include contact information of the service centre or guidance information about the procedures to automatically report a failure.

Thus, according to the bodily fluid analyzing system in accordance with one exemplary embodiment of the present invention, the bodily fluid analyzer (10) that the user has can self-diagnose so that the user can identify an abnormality in the bodily fluid analyzer (10). Therefore, the user can be prevented from being misled about his status by the test and analysis result produced in the malfunctioning bodily fluid analyzer (10).

Moreover, since the management server (20) sends problem solving measures in response to the result of self-diagnosis to the bodily fluid analyzer (10), even when the bodily fluid analyzer (10) has an issue, the user can resolve it according to the problem solving measures, which relieves the user of the burden to visit a service centre when the bodily fluid analyzer (10) has abnormalities.

Hereinafter, in reference with FIG. 2, the bodily fluid analyzer in accordance with one exemplary embodiment of the present invention is illustrated. FIG. 2 shows an outline of composition of the bodily fluid analyzer in accordance with one exemplary embodiment of the present invention.

In reference with FIG. 2, the bodily fluid analyzer (10) in accordance with one exemplary embodiment of the present invention may comprise the communication module (11), the first sensor part (12a), the first operation part (12b), the second sensor part (13a), the second operation part (13b), and the control part (14). However, since not all of the components illustrated in FIG. 2 would be essential, the bodily fluid analyzer (10) may be embodied with a greater or a smaller number of components.

The communication module (11) can communicate with the management server (20) over the external network. For example, the communication module (11) can support communications according to diverse communication specifications including but not limited to 3G and LTE (Long Term Evolution), Wi-Fi, and Bluetooth and it can support diverse wireless/wired communication modes.

Moreover, with a built-in mobile OS, the bodily fluid analyzer (10) in accordance with one exemplary embodiment of the present invention can be run on the bodily fluid analyzer (10) and the types of mobile OS are not limited. Thus, the bodily fluid analyzer (10) in accordance with one exemplary embodiment of the present invention can have connectivity to external networks because it comprises not only the mobile OS but also the communication module (11).

The first sensor part (12a) and the first operation part (12b) can be used for test and analysis of the bodily fluid sample. Specifically, the first sensor (12a) is used as a data entry means of the bodily fluid analyzer (10) and the types are not limited as long as it can be used to test or analyze the bodily fluid sample. For example, the first sensor part (12a) may comprise but is not limited to an optical sensor or a temperature sensor.

Also the first operation part (12b) is used as a means of output of the bodily fluid analyzer (10) and the types of it are not limited as long as it can be used to test or analyze the bodily fluid sample. For example, the first operation part (12b) may comprise but is not limited to an actuator, a light emitting display or a thermo element.

Meanwhile, the second sensor part (13a) and the second operation part (13b) can be used to self-diagnose abnormalities of the first sensor part (12s) or the first operation part (12b). The second sensor part (13a) and the second operation part (13b) and the first sensor part (12a) and the first operation part (12b) may have different purposes of use. Specifically, the first sensor part (12a) and the first operation part (12b) can be used to test and analyze the bodily fluid sample in the bodily fluid analyzer (10). And the second sensor part (12a) and the second operation part (13b) can be used to self-diagnose abnormalities of the first sensor part (12a) and the first operation part (12b) that are used to test and analyze the bodily fluid sample. However, the second sensor part (13a) and the second operation part (13b) are used only for self-diagnosis and therefore, they may have a lower probability of having problems when compared with the first sensor part (12a) and the first operation part (13b).

Specifically, the second sensor part (13a) is used to enter data of the bodily fluid analyzer (10) and the types of it are not limited as long as it can be used to self-diagnose abnormalities of the first sensor part (12a) or the first operation part (12b). For example, the second sensor part (13a) may comprise but is not limited to an optical sensor or a temperature sensor.

And the second operation part (13b) is used as a means of output of the bodily fluid analyzer (10) and the types of it are not limited as long as it can be used to self-diagnose abnormalities of the first sensor part (12a) or the first operation part (12b). For example, the second operation part (13b) may comprise but is not limited to an actuator, a light emitting display or a thermo element.

The control part (14) can control the overall operation of the bodily fluid analyzer (10). For example, the control part (14) can perform control function to be described later via a self-diagnosis application that is run in a mobile OS environment but its function is not limited thereto. However, in this specification, the control part (14) is described with a focus on self-diagnosis function of the bodily fluid analyzer (10).

Hereinafter, how the control part (14) controls self-diagnosis function of the bodily fluid analyzer (10) is described.

First, the control part (14) can operate the first operation part (12b) or the second operation part (13b) based on the predefined conditions and sense the operation status of the first operation part (12b) or the second operation part (13b) by using the second sensor part (13a) or the first sensor part (12a). Specifically, based on the predefined conditions, the control part (14) can operate the first operation part (12b) and sense the operation status of the first operation part (12b) by using the second sensor part (13a) or based on the predefined conditions, it can operate the second operation part (13b) and sense the operation status of the second operation part (13b) by using the first sensor part (12a). Here, the predefined conditions refer to the conditions set for self-diagnosis and for example, they may refer to but are not limited to protocols regarding how to operate the first operation part (12b) or the second operation part (13b).

More specifically, the control part (14) can sense the operation status of the first operation part (12b) to self-diagnose abnormalities of the first operation part (12b). In order to check whether the first operation part (12b) runs as intended, the control part (14), based on the predefined conditions, can operate the first operation part (12b) and sense the operation status of the first operation part (12b).

Also the control part (14) can self-diagnose abnormalities of the sensor function of the first sensor part (12a) by allowing the first sensor part (12a) to sense the operation status of the second operation part (13b). In order to check whether the first sensor part (12a) senses correctly, the control part (14) can operate the second operation part (13b) and sense the operation status of the second operation part (13b) by using the first sensor part (12a), based on the predefined conditions.

Next, the control part (14) can decide whether the first operation part (12b) or the said first sensor part (12a) has abnormalities based on the sensing result of the second sensor part (13a) or the first sensor part (12a).

Specifically, the control part (14) can decide whether the first operation part (12b) has run as intended according to the predefined conditions by using the sensing result of the second sensor part (13a) and by doing so, it can identify abnormalities of the first operation part (12b). If the first operation part (12b) has run as intended according to the predefined conditions, the first operation part (12b) will be found to have no abnormalities and if the first operation part (12b) has not run as intended according to the predefined conditions, the first operation part (12b) will be found to have abnormalities.

Likewise, the control part (14) can determine whether the first sensor part (12a) has sensed correctly the operation status of the second operation part (13b) according to the predefined conditions by using the sensing result of the first sensor part (12a) and by doing so, it can indicate whether the first sensor part (12a) has abnormalities. If the first sensor part (12a) has sensed correctly the operation status of the second operation part (13b) according to the predefined conditions, the first sensor part (12a) will be found to have no abnormalities and if the first sensor part (12a) has not sensed correctly the operation status of the second operation part (13b) according to the predefined conditions, the first sensor part (12a) will be found to have abnormalities.

In addition, if the first operation part (12b) or the first sensor part (12a) is found to have abnormalities, the control part (14) can send the sensing result regarding abnormalities of the first operation part (12b) or the first sensor part (12a) and the sensing result of the second sensor part (13a) or the first sensor part (12a) to the management server (20) via the communication module (11). Here, the aforementioned self-diagnosis result may include but is not limited to the sensing result regarding abnormalities of the first operation part (12b) or the first sensor part (12a) or the sensing result of the second sensor part (13a) or the first sensor part (12a).

Therefore, according to the bodily fluid analyzer (10) in accordance with one exemplary embodiment of the present invention, not only the indication of abnormalities of the bodily fluid analyzer (10) but also the sensing result of the second sensor part (13a) or the first sensor part (12a), which can show the operation status or the sensing result of the corresponding composition, can be sent to the management server (20) and thus the management server (20) can obtain accurate information about the operation status of the bodily fluid analyzer (10) and provide the bodily fluid analyzer (10) with appropriate problem solving measures based on this.

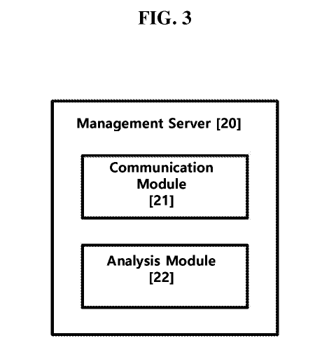

Hereinafter, the management server in accordance with one exemplary embodiment of the present invention is illustrated in reference with FIG. 3. FIG. 3 shows a diagram illustrating an outline of composition of the management server in accordance with one exemplary embodiment of the present invention.

Referring to FIG. 3, the management server (20) in accordance with one exemplary embodiment of the present invention may comprise the communication module (21) and the analysis module (22). However, since not all of components illustrated in FIG. 3 would be essential, the management server (20) can be implemented to have a greater or a smaller number of composition elements.

The communication module (21) can communicate with the bodily fluid analyzer (10) over the external network. For example, the communication module (21) can support diverse communication specifications including but not limited to 3G and LTE (Long Term Evolution), Wi-Fi, and Bluetooth and it can support diverse wireless/wired communication modes.

The analysis module (22) can analyze the self-diagnosis result received from the bodily fluid analyzer (10) to decide whether the bodily fluid analyzer (10) has a problem and identify problem solving measures required to resolve the problem found in the bodily fluid analyzer (10). The problem solving measures are same as the detailed explanations above.

And the problem solving measures identified in the analysis module (22) can be sent to the bodily fluid analyzer (10) via the communication module (21).

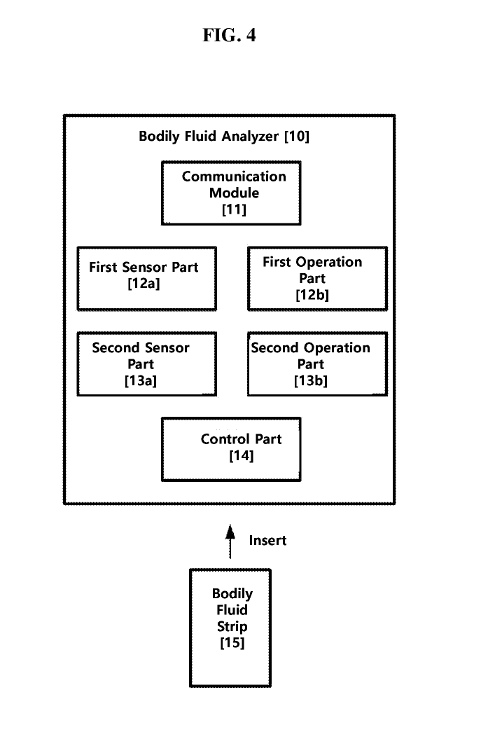

Hereinafter, the bodily fluid analyzer is described in accordance with another exemplary embodiment of the present invention in reference with FIG. 4. However, the focus of explanation is differences of it from the bodily fluid analyzer in accordance with one exemplary embodiment of the present invention. FIG. 4 shows a drawing illustrating an outline of composition of the bodily fluid analyzer in accordance with another exemplary embodiment of the present invention.

According to FIG. 4, the bodily fluid analyzer (10) in accordance with another exemplary embodiment of the present invention can self-diagnose by using the bodily fluid strip (15) that comprises the predefined reference bodily fluid sample. For example, the existence of abnormalities with the bodily fluid analyzer (10) can be determined by checking whether the bodily fluid analyzer (10) tests and analyzes correctly the characteristics of the reference bodily fluid sample.

Specifically, the control part (14), when the bodily fluid strip (15) that comprises the predefined reference bodily fluid sample is inserted to the bodily fluid analyzer (10), can test and analyze the reference bodily fluid sample by using the first sensor part (12a) or the first operation part (12b) and compare the value of test and analysis result with the reference value to detect abnormalities in the first sensor part (12a) or the first operation part (12b).

In other words, when the bodily fluid strip (15) that comprises the predefined reference bodily fluid sample is used, the reference value for the characteristics of the reference bodily fluid sample can be determined in advance. Therefore, the control part (14) can use the first sensor part (12a) or the first operation part (12b) which is for test and analysis of the bodily fluid sample in order to test and analyze the reference bodily fluid sample and can detect abnormalities of the first sensor part (12a) or the first operation part (12b) by comparing the value of the test and analysis result with the reference value.

And the control part (14), if the first operation part (12b) or the first sensor part (12a) is found to have abnormalities, can send the result of abnormality check upon the first operation part (12b) or the first sensor part (12a) and the value of the test and analysis result to the management server (20) via the communication module (11).

And since the bodily fluid analyzer (10) can receive problem solving measures from the management server (20), the user can use it to correct the failure of the bodily fluid analyzer (10).

It is clearly understandable for those having ordinary skill in the art the present invention can be embodied in various forms, other than the exemplary embodiments set forth herein, without changes in its technical idea or essential characteristic. The exemplary embodiments described herein are only for the purpose of exemplifying the present invention in all aspects, not of limiting the scope of the present invention.

1. Bodily fluid analyzer with self-diagnosis function for performing the self-diagnosis, the bodily fluid analyzer comprising:
a first sensor part and a first operation part that are used to test or analyze a bodily fluid sample;
a second sensor part and a second operation part that are used to self-diagnose of problems of the first sensor part or the first operation part; and
control part configured to:
sense an operation status of the first operation part by using the second sensor part after operating the first operation part based on predefined conditions; or
sense an operation status of the second operation part by using the first sensor part after operating the second operation part based on predefined conditions,
wherein the first operation part and the second operation part are means of output of the bodily fluid analyzer.

2. (canceled)

3. The bodily fluid analyzer of claim 1, wherein the control part further configured to:
if the first operation part or the first sensor part is determined to have problems, send a result of determination with the first operation part or the first sensor part and a sensing result of the second sensor part or the first sensor part to the management server.

4. (canceled)

5. The bodily fluid analyzer of claim 1,
wherein a mobile OS system is run on the bodily fluid analyzer, and
wherein the control part is further configured to:
operate the first operation part or the second operation part based on the predefined conditions by operating a self-diagnosis application that is run in a mobile OS environment; and
sense the operation status of the first operation part or the second operation part by using the second sensor part or the first sensor part.

6. The bodily fluid analyzer of claim 1,
wherein the control part is further configured to:
if a bodily fluid strip that comprises a predefined reference bodily fluid sample is inserted into the bodily fluid analyzer, test and analyze the reference bodily fluid sample by using the first sensor part or the first operation part; and
detect problems of the first sensor part or the first operation part by comparing a value of the test and analysis result with a reference value.

7. The bodily fluid analyzer of claim 1,
wherein the first operation part or the second operation part comprises at least one of an actuator, a light emitting display or a thermo element.

8-9. (canceled)

10. The bodily fluid analyzer of claim 3,
wherein the bodily fluid analyzer further comprises a communication module capable of communication with the management server through a external network;
wherein the bodily fluid analyzer receives the result of determination and problem solving measures against to the sensing result from the management server through the communication module; and
wherein the problem solving measures comprise:
guidance information that helps a user of the bodily fluid analyzer operates the bodily fluid analyzer based on the problem solving measures; or
guidance information that helps the user of the bodily fluid analyzer reports a failure of the bodily fluid analyzer based on the problem solving measures.

11. A method for performing self-diagnosis in a bodily fluid analyzer, which comprises: a first sensor part and a first operation part that are used to test or analyze a bodily fluid sample; and a second sensor part and a second operation part that are used to self-diagnose of problems of the first sensor part or the first operation part, wherein the first operation part and the second operation part are means of output of the bodily fluid analyzer, the method comprising:
operating the first operation part or the second operation part;
sensing, if the first operation part is operated, an operation status of the first operation part by using the second sensor part; and
sensing, if the second operation part is operated, an operation status of the second operation part by using the first sensor part.

12. The method of claim 11, further comprising:
respectively determining, according to a sensing result of the second sensor part of the first sensor part, problems of the first operation part or the first sensor part; and
sending, if the first operation part or the first sensor part is determined to have problems, a result of determination with the first operation part or the first sensor part and the sensing result of the second sensor part or the first sensor part to the management server.

13. The method of claim 11, wherein a mobile OS system is run on the bodily fluid analyzer, and
wherein the performing the self-diagnosis comprises performing the self-diagnosis of the bodily fluid analyzer by operating a self-diagnosis application that is run in a mobile OS environment.

14. The method of claim 11, further comprising:
inserting a bodily fluid strip that comprises a predefined reference bodily fluid sample into the bodily fluid analyzer;
testing and analyzing the reference bodily fluid sample by using the first sensor part or the first operation part; and
detecting problems of the first sensor part or the first operation part by comparing a value of the test and analysis result with a reference value.

15. The method of claim 11, wherein the first operation part or the second operation part comprises at least one of an actuator, a light emitting display or a thermo element.

16. The method of claim 12,
wherein the bodily fluid analyzer further comprises a communication module capable of communication with the management server through a external network,
wherein the performing self-diagnosis further comprises receiving the result of determination and problem solving measures against to the sensing result from the management server through the communication module; and
wherein the problem solving measures comprise:
guidance information that helps a user of the bodily fluid analyzer operates the bodily fluid analyzer based on the problem solving measures; or
guidance information that helps the user of the bodily fluid analyzer reports a failure of the bodily fluid analyzer based on the problem solving measures.
